(19) United States
(12) Patent Application Publication
(10) Pub. No.: US 2023/0009249 A1
(43) Pub. Date: Jan. 12, 2023

(54) EXPANDABLE TRANSITION ELEMENT FOR A TRANSCATHETER DELIVERY DEVICE

(71) Applicant: Edwards Lifesciences Corporation, Irvine, CA (US)

(72) Inventors: Oren Cohen, Modi'in (IL); Tomer Saar, Pardes Hanna-Karkur (IL); Tamir S. Levi, Zikhron Yaakov (IL); Yair A. Neumann, Moshav Sede Varburg (IL); David Maimon, Atlit (IL)

(73) Assignee: Edwards Lifesciences Corporation, Irvine, CA (US)

(21) Appl. No.: 17/732,185

(22) Filed: Apr. 28, 2022

Related U.S. Application Data
(63) Continuation of application No. PCT/US2020/055546, filed on Oct. 14, 2020.
(60) Provisional application No. 62/928,973, filed on Oct. 31, 2019.

(51) Int. Cl. A61F 2/24

(52) U.S. Cl. CPC A61F 2/2418; A61F 2/2433; A61F 2/2436

(57) ABSTRACT

A transcatheter delivery system including an expandable transition element is disclosed. As one example, an assembly may comprise a prosthetic valve and a delivery device. The delivery device may comprise an outer shaft with a distal end portion forming a sheath adapted to enclose the prosthetic valve therein in a radially compressed configuration; an inner shaft arranged within the outer shaft and including a nosecone arranged at a distal end of the inner shaft, the nosecone arranged outside of the outer shaft, at the distal end portion of the outer shaft; and an expandable transition element adapted to expand from a non-expanded state within the outer shaft to an expanded state outside the outer shaft, wherein, in the expanded state, the transition element forms a continuous transition from the nosecone to the prosthetic valve when the sheath is moved away from the nosecone to uncover the prosthetic valve.

### CROSS REFERENCE TO RELATED APPLICATIONS

This application is a continuation of PCT Patent Application No. PCT/US2020/055546, entitled “EXPANDABLE TRANSITION ELEMENT FOR A TRANSCATHETER DELIVERY DEVICE,” filed Oct. 14, 2020, which claims the benefit of U.S. Provisional Application No. 62/928,973 entitled “EXPANDABLE TRANSITION ELEMENT FOR A TRANSCATHETER DELIVERY DEVICE,” filed Oct. 31, 2019, all of which are incorporated by reference herein in their entirety.

### FIELD

The present disclosure concerns embodiments of assemblies, and related methods, for providing a more continuous transition, via a transition element, between a prosthetic medical device and a nosecone of a delivery apparatus adapted to deliver the prosthetic medical device to a target implantation site.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device (e.g., delivery apparatus), proximate to a nosecone of the delivery device, and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically expandable” prosthetic heart valves. The actuator typically takes the form of pull cables, sutures, wires and/or shafts that are configured to transmit expansion forces from a handle of the delivery apparatus to the prosthetic valve.

In some embodiments, after the prosthetic valve is deployed from the sheath of the delivery device, but prior to being actively expanded via actuators of the delivery device, the prosthetic valve may assume a partially expanded (e.g., non-compressed) diameter that is larger than its fully compressed diameter (after being crimped) and smaller than its fully expanded diameter (after being expanded via actuators of the delivery device). As a result of this expansion in diameter, a gap may form between the nosecone of the delivery device and a distal end of the prosthetic valve. This gap creates a discontinuity between the prosthetic valve and the nosecone which may make it difficult to reposition the valve at the target implantation site. For example, in some embodiments, the gap may cause the prosthetic valve to come into unwanted contact with the patient's anatomy during repositioning of the valve. Accordingly, improvements in delivery devices which reduce gap formation between a nosecone of the delivery device and the prosthetic valve (after deployment from a sheath of the delivery device, in some examples), are desirable.

### SUMMARY

Disclosed herein are assemblies including a prosthetic valve and delivery apparatus and related methods for delivering a prosthetic valve to and implanting the prosthetic valve at a target implantation site with a delivery apparatus. The delivery apparatuses (which can also be referred to herein as delivery devices) can be used to deliver an implantable medical device, such as a prosthetic heart valve, to a target site in a patient, such as a heart. In some embodiments, delivery apparatuses can be a component of a delivery system (e.g., an endovascular or transcatheter delivery system) that can be used to deliver a prosthetic heart valve or other implantable medical device.

In some embodiments, the delivery apparatus may be configured with an expandable transition element that is arranged, in a non-expanded (e.g., compressed) state, within an outer shaft of the delivery apparatus during delivery (e.g., maneuvering) of the delivery apparatus to the target implantation site. The transition element may be adapted to expand from the non-expanded state within the outer shaft to an expanded state outside the outer shaft, where, in the expanded state, the transition element forms a continuous transition from a nosecone of the delivery apparatus to the prosthetic valve when a distal end of the outer shaft is moved away from the nosecone to uncover the prosthetic valve. The expandable transition element may be one of an inflatable balloon, a pre-inflated balloon, a compressible element (such as a sponge), and a mechanical element (having an expandable frame).

In one representative embodiment, an assembly includes a prosthetic valve and a delivery apparatus. The delivery apparatus includes an outer shaft with a distal end portion forming a sheath adapted to enclose the prosthetic valve therein in a radially compressed configuration; an inner shaft arranged within the outer shaft and including a nosecone arranged at a distal end of the inner shaft, the nosecone arranged outside of the outer shaft, at the distal end portion of the outer shaft; and an expandable transition element adapted to expand from a non-expanded state within the outer shaft to an expanded state outside the outer shaft, wherein, in the expanded state, the transition element forms a continuous transition from the nosecone to the prosthetic valve when the sheath is moved away from the nosecone to uncover the prosthetic valve.

In some embodiments, the delivery apparatus further comprises at least one actuator assembly arranged within the outer shaft and releasably coupled to the prosthetic valve.

In some embodiments, the transition element is a balloon.

In some embodiments, the balloon is an inflatable balloon that is inflatable from a deflated state prior to removal of the prosthetic valve from the sheath to an inflated state after removal of the prosthetic valve from the sheath. Further, in some embodiments, when the balloon is in the deflated state, it is arranged within an interior of the sheath, between the nosecone and a distal end of the prosthetic valve, in the radially compressed configuration. In some embodiments, when the balloon is in the inflated state, it is arranged exterior to the outer shaft and between the nosecone and a distal end of the prosthetic valve.

In some embodiments, the balloon is a compliant balloon formed from an elastic material and is configured to be inflated to a desired size within a range of possible sizes based on a size of the prosthetic valve.

In some embodiments, the balloon is a semi-compliant balloon comprising Pebax.

In some embodiments, the balloon is a noncompliant balloon formed from a non-elastic material and is configured to expand to a predetermined size when fully inflated, where the predetermined size is selected based on a size of the prosthetic valve.

In some embodiments, the balloon is a pre-inflated balloon, pre-inflated to an expanded state, that passively transitions between a compressed state when positioned within the sheath to the expanded state when the sheath is moved away from the balloon.

In some embodiments, the transition element is a compressible element including one or more of a compressible foam and a sponge. In some embodiments, a proximal end of the compressible element is tapered inward toward a central longitudinal axis of the assembly.

In some embodiments, the transition element is an expandable, mechanical element. In some embodiments, the mechanical element comprises an expandable frame including a plurality of arms, wherein each arm of the plurality of arms includes a distal end attached to the nosecone and a proximal end that is unattached to the delivery apparatus and adapted to expand from a compressed state to an expanded state. In some embodiments, the mechanical element further comprises a cover surrounding the plurality of arms, around a circumference of the expandable frame. In some embodiments, the mechanical element further comprises a compression mechanism configured to re-compress the frame from the expanded state to the compressed state.

In some embodiments, in the expanded state, a proximal end of the transition element contacts a distal end of the prosthetic valve and a distal end of the transition element contacts a proximal end of the nosecone.

In some embodiments, a distal end of the transition element is attached to a proximal end of the nosecone.

In another representative embodiment, a method includes advancing a delivery apparatus of a transcatheter delivery system to a target implantation site in a patient, the delivery apparatus including an outer shaft with a distal end portion forming a sheath enclosing a radially compressed prosthetic valve therein, proximate to a proximal end of a nosecone of the delivery apparatus; after reaching the target implantation site, moving the distal end portion of the outer shaft away from the nosecone, in an axial direction, to uncover the prosthetic valve; and expanding a transition element of the delivery apparatus in a space formed between the proximal end of the nosecone and a distal end of the prosthetic valve.

In some embodiments, the prosthetic valve expands to a partially expanded state upon moving the distal end portion of the outer shaft away from the nosecone.

In some embodiments, the method can further include, after expanding the transition element, repositioning the prosthetic valve, in the partially expanded state, at the target implantation site.

In some embodiments, the method can further include, after repositioning the prosthetic valve, actively expanding, in a radial direction, the prosthetic valve to a radially expanded state.

In some embodiments, actively expanding the prosthetic valve includes actively expanding the prosthetic valve via one or more actuator assemblies of the delivery apparatus, the one or more actuator assembly extending from an interior of the outer shaft and coupled to the prosthetic valve.

In some embodiments, the transition element is an inflatable balloon and expanding the transition element includes inflating the inflatable balloon from a deflated state to an inflated state.

In some embodiments, the inflatable balloon is a compliant balloon formed from an elastic material and inflating the inflatable balloon from the deflated state to the inflated state includes inflating the inflatable balloon to a desired size within a range of possible sizes that is based on a size of the prosthetic valve.

In some embodiments, the inflatable balloon is a semi-compliant balloon comprising Pebax and inflating the inflatable balloon from the deflated state to the inflated state includes inflating the inflatable balloon to a desired size within a range of possible sizes that is based on a size of the prosthetic valve.

In some embodiments, the inflatable balloon is a noncompliant balloon formed from a non-elastic material and inflating the inflatable balloon from the deflated state to the inflated state includes inflating the inflatable balloon to a predetermined size that is selected based on a size of the prosthetic valve.

In some embodiments, a distal end of the inflatable balloon is attached to the proximal end of the nosecone.

In some embodiments, the transition element is a pre-inflated balloon and expanding the transition element includes passively expanding the pre-inflated balloon from a radially compressed state to a radially expanded state, wherein the pre-inflated balloon assumes its pre-inflated size when in the radially expanded state.

In some embodiments, the transition element is a compressible element including one of a compressible foam and a sponge material and expanding the transition element includes passively expanding the compressible element from a compressed state to an expanded, non-compressed state, wherein the compressible element is in its resting state when in the expanded state.

In some embodiments, the transition element is a mechanical element comprising an expandable frame having a distal end coupled to the nosecone and expanding the transition element includes expanding a proximal end of the expandable frame from a compressed state to an expanded state.

In another representative embodiment, an assembly can include a mechanically expandable prosthetic valve including a distal end and a proximal end and a delivery apparatus. The delivery apparatus can include an outer shaft with a distal end portion forming a sheath adapted to enclose the prosthetic valve therein in a radially compressed configuration; at least one actuator assembly arranged within the outer shaft and releasably coupled to the prosthetic valve; an inner shaft arranged within the outer shaft and including a nosecone arranged at a distal end of the inner shaft, the nosecone arranged outside of the outer shaft and proximate to the distal end of the prosthetic valve; and an expandable transition element adapted to expand from a non-expanded state within the outer shaft to an expanded state outside the outer shaft, wherein, in the expanded state, the transition element forms a continuous transition from a proximal end of the nosecone to the distal end of the prosthetic valve when the sheath is moved away from the nosecone to uncover the prosthetic valve.

In some embodiments, a distal end of the transition element is attached to the proximal end of the nosecone.

In some embodiments, the transition element is an inflatable balloon adapted to be inflated from a deflated state prior to removal of the prosthetic valve from the sheath to an inflated state after removal of the prosthetic valve from the sheath.

In some embodiments, the balloon is a compliant balloon formed from an elastic material and is configured to be inflated to a desired size within a range of possible sizes based on a size of the prosthetic valve.

In some embodiments, the balloon is a semi-compliant balloon comprising Pebax.

In some embodiments, the balloon is a noncompliant balloon formed from a non-elastic material and is configured to expand to a predetermined size when fully inflated, wherein the predetermined size is selected based on a size of the prosthetic valve.

In some embodiments, the transition element is a pre-inflated balloon, pre-inflated to an expanded state, that passively transitions between a compressed state when positioned within the sheath to the expanded state when the sheath is moved away from the balloon.

In some embodiments, the pre-inflated balloon is pre-filled with saline.

In some embodiments, the pre-inflated balloon is pre-filled with a hydrogel.

In some embodiments, the transition element is a compressible element including one or more of a compressible foam and a sponge.

In some embodiments, the transition element is an expandable, mechanical element comprising an expandable frame including a plurality of arms, wherein each arm of the plurality of arms includes a distal end attached to the nosecone and a proximal end that is unattached to the delivery apparatus and adapted to expand from a compressed state when positioned within the sheath to an expanded state when the sheath is moved away from the mechanical element.

In some embodiments, when in the expanded state, the transition element tapers in diameter from the distal end of the prosthetic valve to the proximal end of the nosecone.

In another representative embodiment, an assembly includes a prosthetic valve and a delivery apparatus. The delivery apparatus includes an outer shaft with a distal end portion forming a sheath adapted to enclose the prosthetic valve therein in a radially compressed configuration; an inner shaft arranged within the outer shaft and including a nosecone arranged at a distal end of the inner shaft, the nosecone arranged outside of the outer shaft, wherein the outer shaft and the inner shaft are configured to move axially relative to one another to move the nosecone away from the distal end portion of the outer shaft and uncover the prosthetic valve; and an expandable transition element disposed between the prosthetic valve and the nosecone, the expandable transition element adapted to expand from a non-expanded state within the outer shaft to an expanded state outside the outer shaft, wherein the transition element is in the non-expanded state when the sheath covers the prosthetic valve and the transition element and is in the expanded state when the sheath is moved away from the nosecone to uncover the prosthetic valve and wherein, in the expanded state, the transition element forms a continuous transition from the nosecone to the prosthetic valve.

In some embodiments, a distal end of the transition element is attached to a proximal end of the nosecone.

In some embodiments, the delivery apparatus further comprises at least one actuator assembly arranged within the outer shaft and releasably coupled to the prosthetic valve.

In some embodiments, the at least one actuator assembly is configured to radially expand the prosthetic heart valve.

In some embodiments, the transition element is a balloon.

In some embodiments, the balloon is an inflatable balloon that is configured to receive an inflation fluid and inflate from a deflated state to an inflated state.

In some embodiments, when the balloon is in the deflated state, it is arranged within an interior of the sheath, between the nosecone and a distal end of the prosthetic valve, in the radially compressed configuration.

In some embodiments, when the balloon is in the inflated state, it is arranged exterior to the outer shaft and between the nosecone and a distal end of the prosthetic valve.

In some embodiments, the balloon is a compliant balloon formed from an elastic material and is configured to be inflated to a desired size within a range of possible sizes based on a size of the prosthetic valve.

In some embodiments, the balloon is a semi-compliant balloon comprising Pebax.

In some embodiments, the balloon is a noncompliant balloon formed from a non-elastic material and is configured to expand to a predetermined size when fully inflated, wherein the predetermined size is selected based on a size of the prosthetic valve.

In some embodiments, the balloon is a pre-inflated balloon, pre-inflated to an expanded state, that passively transitions between a compressed state when positioned within the sheath to the expanded state when the sheath is moved away from the balloon.

In some embodiments, the transition element is a compressible element including one or more of a compressible foam and a sponge.

In some embodiments, a proximal end of the compressible element is tapered inward toward a central longitudinal axis of the assembly.

In some embodiments, the transition element is an expandable, mechanical element.

In some embodiments, the mechanical element comprises an expandable frame including a plurality of arms, where each arm of the plurality of arms includes a distal end attached to the nosecone and a proximal end that is unattached to the delivery apparatus and adapted to expand from a compressed state to an expanded state.

In some embodiments, the mechanical element further comprises a cover surrounding the plurality of arms, around a circumference of the expandable frame.

In some embodiments, the mechanical element further comprises a compression mechanism configured to re-compress the frame from the expanded state to the compressed state.

In some embodiments, in the expanded state, a proximal end of the transition element contacts a distal end of the prosthetic valve and a distal end of the transition element contacts a proximal end of the nosecone.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary embodiment of a prosthetic heart valve.

FIG. 2 is a perspective view of a portion of another exemplary embodiment of a prosthetic heart valve.

FIG. 3 is a side view of the frame of the prosthetic heart valve of FIG. 2, shown in a radially collapsed configuration.

FIG. 4 is a side view of the frame of the prosthetic heart valve of FIG. 2, shown in a radially expanded configuration.

FIG. 5 is a side view of an embodiment of a prosthetic valve delivery apparatus.

FIGS. 6A-6C are sides views of a portion of the delivery apparatus of FIG. 5 in various stages of a prosthetic valve placement procedure.

FIGS. 7A-7D show side views of a portion of a delivery apparatus including a transition element adapted to be positioned between a nosecone of the delivery apparatus and a non-compressed prosthetic valve, where the transition element comprises a balloon.

FIGS. 8A-8D show side views of a portion of a delivery apparatus including a transition element adapted to be positioned between a nosecone of the delivery apparatus and a non-compressed prosthetic valve, where the transition element comprises a compressible element.

FIGS. 9A-9C show side views of a portion of a delivery apparatus including a transition element adapted to be positioned between a nosecone of the delivery apparatus and a non-compressed prosthetic valve, where the transition element comprises a mechanical element.

FIG. 10 is a flow chart of a method for delivering a prosthetic valve to a target implantation site with a delivery apparatus including an expandable transition element, according to an embodiment.

### DETAILED DESCRIPTION

Described herein are examples of prosthetic valves, delivery apparatus (or devices) configured to deliver prosthetic valves to target implantation locations within a body, and methods for delivering a prosthetic valve to and implanting the prosthetic valve at a target implantation site with a delivery apparatus. The prosthetic valves (e.g., prosthetic heart valves) may include a frame including a proximal end and distal end. As used herein, the “distal end” of the frame may refer to the end of the frame that is positioned proximate and/or adjacent to a distal shoulder/nosecone of a delivery apparatus when arranged within an outer shaft of the delivery apparatus. For example, the distal end may be oriented further downstream than the proximal end of the frame when the delivery apparatus in which the prosthetic valve is arranged is being advanced through a lumen of a patient, toward a target implantation site.

The delivery apparatus may include an outer shaft with a distal end portion forming a sheath (or capsule) adapted to enclose the prosthetic valve therein in a radially compressed configuration during advancement of the delivery apparatus to the target implantation site. The delivery apparatus may further include an inner shaft arranged within the outer shaft and including a nosecone arranged at a distal end of the inner shaft, the nosecone arranged outside of the outer shaft, at the distal end portion of the outer shaft (while the outer shaft is covering the prosthetic valve). In some embodiments, the delivery apparatus may further include an expandable transition element adapted to expand from a non-expanded state within the outer shaft to an expanded state outside the outer shaft. In the expanded state, the transition element may form a continuous transition, in an axial direction relative to a central longitudinal axis of the delivery apparatus, from the nosecone to the prosthetic valve when the sheath is moved away from the nosecone to uncover the prosthetic valve. As a result, the prosthetic valve may be more easily repositioned via the delivery apparatus at the target implantation site, without causing unwanted contact between sides of the patient's anatomy and the prosthetic valve (which may cause damage to the anatomy or valve in some cases).

The prosthetic valves disclosed herein can be radially compressible and expandable between a radially compressed configuration and a radially expanded configuration. Thus, the prosthetic valves can be crimped on an implant delivery apparatus (e.g., device) in the radially compressed configuration during delivery, and then expanded to the radially expanded configuration once the prosthetic valve reaches the implantation site.

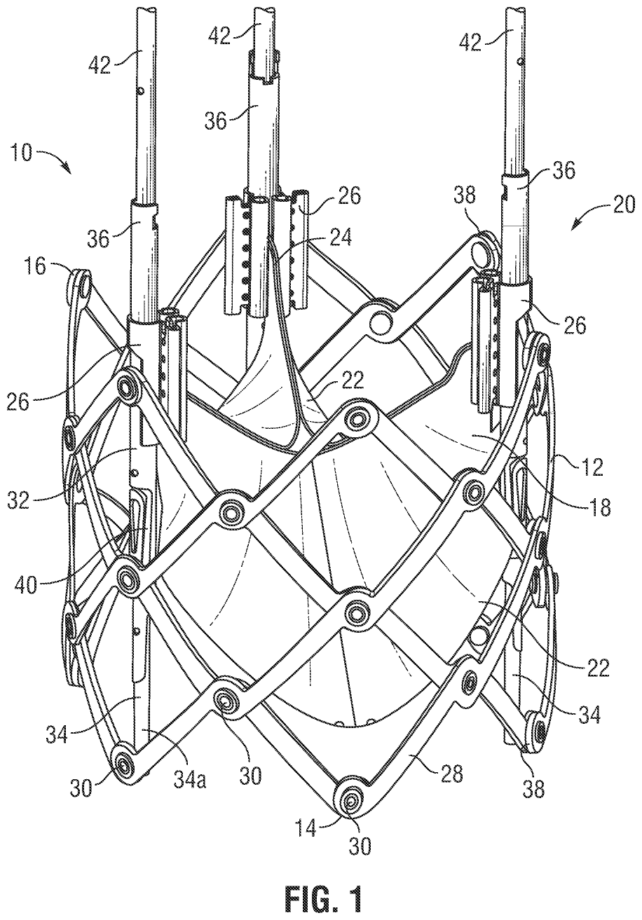

FIG. 1 shows an exemplary prosthetic valve 10, according to one embodiment. The prosthetic valve 10 can be radially compressible and expandable between a radially compressed configuration for delivery into a patient (see e.g., FIG. 3) and a radially expanded configuration (see e.g., FIGS. 1 and 4). In particular embodiments, the prosthetic valve 10 can be implanted within the native aortic annulus, although it also can be implanted at other locations in the heart, including within the native mitral valve, the native pulmonary valve, and the native tricuspid valve. The prosthetic valve 10 can include an annular stent or frame 12 having a first end 14 and a second end 16.

In the depicted embodiments, the first end 14 is an inflow end and the second end 16 is an outflow end. The outflow end 16 can be coupled to a delivery apparatus for delivering and implanting the prosthetic valve within the native aortic valve is a transfemoral, retrograde delivery approach. Thus, in the delivery configuration of the prosthetic valve, the outflow end 16 is the proximal-most end of the prosthetic valve. In other embodiments, the inflow end 14 can be coupled to the delivery apparatus, depending on the particular native valve being replaced and the delivery technique that is used (e.g., trans-septal, transapical, etc.). For example, the inflow end 14 can be coupled to the delivery apparatus (and therefore is the proximal-most end of the prosthetic valve in the delivery configuration) when delivering the prosthetic valve to the native mitral valve via a trans-septal delivery approach.

The prosthetic valve 10 can also include a valvular structure 18 which is coupled to the frame 12 and configured to regulate the flow of blood through the prosthetic valve 10 from the inflow end to the outflow end. The prosthetic valve 10 can further include a plurality of actuators 20 mounted to and equally spaced around the inner surface of the frame 12. Each of the actuators 20 can be configured to form a releasable connection with one or more respective actuators of a delivery apparatus, as further described below.

The valvular structure 18 can include, for example, a leaflet assembly comprising one or more leaflets 22 (three leaflets 22 in the illustrated embodiment) made of a flexible material. The leaflets 22 of the leaflet assembly can be made from in whole or part, biological material, bio-compatible synthetic materials, or other such materials. Suitable biological material can include, for example, bovine pericardium (or pericardium from other sources). The leaflets 22 can be arranged to form commissures 24, which can be, for example, mounted to respective actuators 20. Further details regarding transcatheter prosthetic heart valves, including the manner in which the valvular structure can be coupled to the frame 12 of the prosthetic valve 10, can be found, for example, in U.S. Pat. Nos. 6,730,118, 7,393,360, 7,510,575, 7,993,394, and 8,652,202, and U.S. Patent Application Publication No. 2018/0325665, all of which are incorporated herein by reference in their entireties.

In some embodiments, the prosthetic valve 10 can include a plurality of commissure support elements configured as commissure clasps or clamps 26. In the illustrated configuration, the prosthetic valve includes a commissure clamp 26 positioned at each commissure 24 and configured to grip adjacent portions of two leaflets 22 at each commissure 24, at a location spaced radially inwardly of the frame 12. Each clamp 26 can be mounted on an actuator 20 as shown. In alternative embodiments, the commissure supports elements (such as clamps 26) can be mounted to the struts 28 of the frame, or alternatively, the commissures 24 can be mounted (e.g., sutured) directly to the struts of the frame. Further details of the commissure clamps 26 and other techniques for mounting the commissures of a valve assembly to a frame can be found in U.S. Patent Application Publication No. 2018/0325665.

Although not shown, the prosthetic valve 10 can also include one or more skirts or sealing members. For example, the prosthetic valve 10 can include an inner skirt mounted on the inner surface of the frame. The inner skirt can function as a sealing member to prevent or decrease perivalvular leakage, to anchor the leaflets 22 to the frame, and/or to protect the leaflets against damage caused by contact with the frame during crimping and during working cycles of the prosthetic valve. The prosthetic valve 10 can also include an outer skirt mounted on the outer surface of the frame 12. The outer skirt can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

The frame 12 can be made of any of various suitable materials, such as stainless steel, a cobalt chromium alloy, or a nickel titanium alloy (“NiTi”), for example Nitinol. Referring again to FIG. 1, as shown, the frame 12 can include a plurality of interconnected struts 28 arranged in a lattice-type pattern. The struts 28 are shown as positioned diagonally, or offset at an angle relative to, and radially offset from, a longitudinal axis of the prosthetic valve 10 when the prosthetic valve 10 is in the expanded configuration. In other implementations, the struts 28 can be offset by a different amount than depicted in FIG. 1, or some or all of the struts 28 can be positioned parallel to the longitudinal axis of the prosthetic valve 10.

In the illustrated embodiment, the struts 28 are pivotably coupled to one another at one or more pivot joints along the length of each strut. For example, in the illustrated configuration, each of the struts 28 can be formed with apertures (see e.g., apertures 114 in FIG. 4) at opposing ends of the strut and apertures spaced along the length of the strut. Respective hinges can be formed at the locations where struts 28 overlap each other via fasteners or pivot members, such as rivets or pins 30 that extend through the apertures. The hinges can allow the struts 28 to pivot relative to one another as the frame 12 is radially expanded or compressed, such as during assembly, preparation, or implantation of the prosthetic valve 10.

In some embodiments, the frame 12 can be constructed by forming individual components (e.g., the struts and fasteners of the frame) and then mechanically assembling and connecting the individual components together. In other embodiments, the struts 28 are not coupled to each other with respective hinges but are otherwise pivotable or bendable relative to each other to permit radial expansion and contraction of the frame 12. For example, the frame 12 can be formed (e.g., via laser cutting, electroforming or physical vapor deposition) from a single piece of material (e.g., a metal tube). Further details regarding the construction of the frame and the prosthetic valve are described in U.S. patent application Ser. Nos. 15/831,197; 62/515,437; 62/548,855, all of which are incorporated herein by reference. Additional examples of expandable prosthetic valves that can be used with the delivery apparatuses disclosed herein are described in U.S. Publication No. 2015/0135506 and 2014/0296962, which are incorporated herein by reference.

Referring still to FIG. 1, in some embodiments, the prosthetic valve 10 can comprise one or more actuators 20 configured to produce radial expansion and compression of the frame. The one or more actuators in the illustrated embodiment comprise one or more push-pull mechanisms 32 coupled to the frame 12. In the illustrated embodiment, the prosthetic valve 10 has three push-pull mechanisms 32, however, in other embodiments a greater or fewer number of push-pull mechanisms 32 can be used.

Each push-pull mechanism 32 can generally comprise an inner member 34, such as an inner tubular member, and an outer member 36 disposed about the inner member 34. The inner members 34 and the outer members 36 can be movable longitudinally relative to each other in a telescoping manner to radially expand and contract the frame 12, as further described in U.S. patent application Ser. Nos. 62/430,810, 15/831,197; 15/978,459, which are incorporated herein by reference. The inner members 34 can be, for example, rods, cables, wires, or tubes. The outer members 36 can be, for example, tubes or sheaths having sufficient rigidity such that they can apply a distally directed force to the frame without bending or buckling.

The inner members 34 can have distal end portions 34a coupled to the inflow end 14 of the frame 12 (e.g., with a coupling element such as a pin member 30). In the illustrated embodiment, each of the inner members 34 are coupled to the frame at respective apices 38 at the inflow end 14 of the frame 12. For example, the distal end portion 34a of each inner member 34 can be pivotably connected to the rivet or pin 30 that connects the two struts at the adjacent apex 38. The outer members 36 can be coupled to apices 38 at the outflow end 16 of the frame 12 at, for example, a mid-portion of the outer member 36, as shown in FIG. 1, or at a proximal end portion of the outer member, as desired. The outer members 36 can be pivotably connected to the rivet or pin 30 that connects the two struts at the adjacent apex 38.

The inner member 34 and the outer member 36 can telescope relative to each other between a fully contracted state (corresponding to a fully radially expanded state of the prosthetic valve) and a fully extended state (corresponding to a fully radially compressed state of the prosthetic valve). In the fully extended state, the inner member 34 is fully extended from the outer member 36. In this manner, the push-pull mechanisms 32 allow the prosthetic valve to be fully expanded or partially expanded to different diameters and retain the prosthetic valve in the partially or fully expanded state. It should be understood that the inner members 34 and the outer members 36 can be coupled to other locations on the frame to produce radial compression and expansion of the frame, so long as the inner member and outer member of each actuator are coupled at axial spaced pivot joints of the frame.

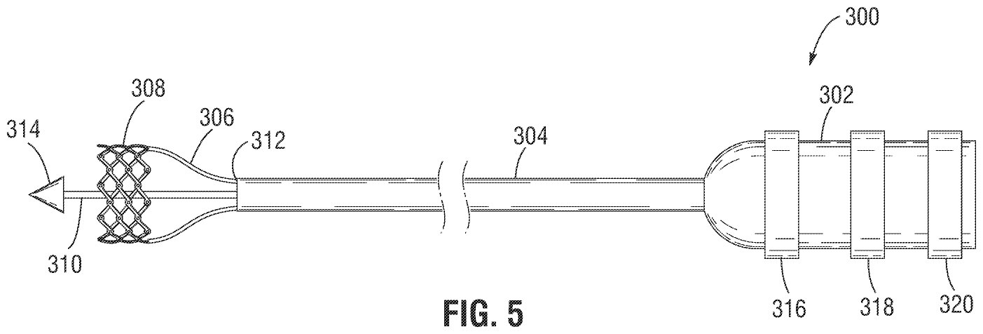

In use, a delivery apparatus, such as example delivery apparatus (e.g., device) 300 shown in FIG. 5, as described further below, can be releasably coupled to the push-pull mechanisms 32 of prosthetic valve 10. For example, the delivery apparatus can have one or more actuator assemblies that are releasably coupled to respective push-pull mechanisms 32 of the prosthetic valve. The actuators (e.g., actuator assemblies) of the delivery apparatus can be configured to transfer pushing and/or pulling forces from a handle of the delivery apparatus to the push-pull mechanisms 32 of the prosthetic valve. Each of the actuator assemblies of the delivery apparatus can include an inner member 42 that is releasably coupled to a respective inner member 34 of a push-pull mechanism 32. Each actuator assembly of the delivery apparatus can also include an outer member (not shown) that is releasably coupled to a respective outer member 36 of a push-pull mechanism 32.

Once coupled to the delivery apparatus, the prosthetic valve 10 can then be radially collapsed (see e.g., FIG. 3) and the distal end portion of the delivery apparatus, along with the radially collapsed valve, can be inserted into a patient. Once the prosthetic valve 10 is at the desired implantation site, the prosthetic valve can be radially expanded (see e.g., FIG. 4). In some embodiments, as shown in FIG. 1, the push-pull mechanisms 32 can comprise one or more locking mechanisms 40, allowing the frame 12 to maintain an expanded diameter after the prosthetic valve is released from the delivery apparatus. Additional details of the locking mechanism can be found in U.S. Patent Application Publication No. 2018/0325665.

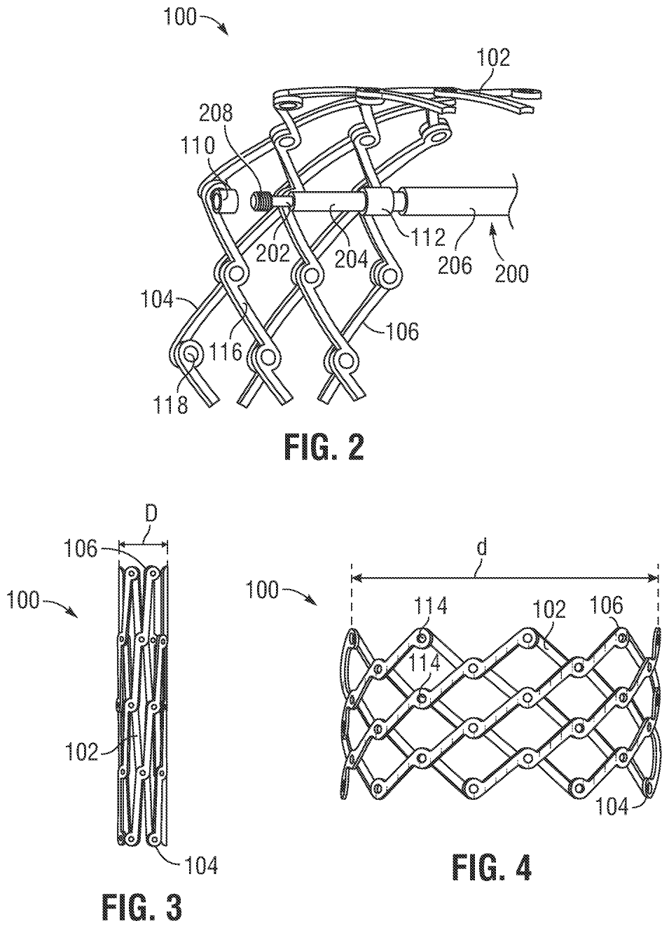

FIG. 2 illustrates a medical assembly, according to another embodiment. The assembly comprises a prosthetic valve 100 and one or more linear actuator assemblies 200 (one shown in FIG. 2) releasably coupled to the prosthetic valve. The prosthetic valve 100 comprises a frame 102. The prosthetic valve 100 can include a valvular structure (e.g., including leaflets) 18 and inner and/or outer skirts as previously described, although these components are omitted for purposes of illustration. The frame 102 comprises a plurality of struts 116 formed with apertures 114 (see FIG. 4) and pivot members 118 (e.g., pins or rivets) connecting the struts to each other form a plurality of pivot joints. The frame 102 can have the same construction as the frame 12, except that the frame 102 includes struts 116 that are longer than struts 28 of frame 12. The longer struts 116 form more pivot joints along the length of each strut and more openings or cells of the frame compared to the struts 28.

FIGS. 3-4 illustrate the bare frame 102 (without the leaflets and other components) of the prosthetic valve 100 for purposes of illustrating expansion of the prosthetic valve from the radially compressed configuration to the radially expanded configuration. FIG. 3 shows the frame 102 in the radially compressed configuration (having diameter D), and FIG. 4 shows the frame 102 in the fully radially expanded configuration (having diameter d). The prosthetic valve 100 in the illustrated configuration can be radially expanded by maintaining the first end 104 of the frame 102 at a fixed position while applying a force in the axial direction against the second end 106 toward the first end 104. Alternatively, the prosthetic valve 100 can be expanded by applying an axial force against the first end 104 while maintaining the second end 106 at a fixed position, or by applying opposing axial forces to the first and second ends 104, 106, respectively.

The one or more actuator assemblies 200 can be components of a delivery apparatus (e.g., the delivery apparatus 300 of FIG. 5) and are configured to produce radial expansion and compression of the frame 102. FIG. 2 shows a linear actuator assembly 200 in the process of being disconnected from the frame 102 after the frame has been radially expanded. As shown, the actuator assembly 200 can include an inner actuator member 202 (which can also be referred to as an actuation member), a cover tube 204 extending co-axially over the actuator member 202, a support tube or pusher member 206 extending co-axially over the cover tube 204, a threaded screw 208. The actuator member 202 can be, for example, a rod, cable, or wire. The actuator member 202 can be connected at its distal end to the threaded screw 208 such that rotation of the actuator member 202 causes rotation of the threaded screw 208. The proximal end of the actuator member 202 can be connected to a handle or other control device (not shown) of the delivery apparatus that a doctor or operator of the delivery apparatus can use to rotate the actuator member 202. Similarly, the proximal ends of each cover tube 204 and each support tube 206 can be connected to the handle. For each actuator assembly 200, a pair of a threaded nut or sleeve 110 and a stopper 112 can be affixed to the frame at axially spaced locations, such as at locations at or adjacent the distal and proximal ends of the frame.

The screw 208 has an externally threaded surface that can engage an internally threaded surface of the sleeve 110, which is affixed to the frame 102, such as at the distal end of the frame. When the actuator member 202 is rotated to screw the screw 208 into the sleeve 110, the actuator member 202 becomes connected to the distal end of the frame 102 such that proximal or distal motion of the actuator member 202 causes proximal or distal motion, respectively, of the distal end of the frame 102.

The cover tube 204 annularly surrounds the actuator member 202. The cover tube 204 can be connected to the actuator member 202 such that the actuator member 202 and the cover tube 204 rotate together and move axially together. The actuator member 202 and the cover tube 204 extend through the stopper 112, which can be affixed to a proximal end of the frame. The support tube 206 annularly surrounds the cover tube 154. The stopper 112 has an annular inner surface with an inner diameter larger than the outer diameter of the cover tube 204 and the screw 208 such that the cover tube 204 and the screw 208 can be retracted through the stopper 112 as the frame 102 is expanded and once the actuator is retracted proximally by the user to disconnect it from the frame. The stopper 112 is sized to abut or engage the distal end of the support tube 206 such that the support tube 206 is prevented from moving distally beyond the stopper 112.

In operation, prior to implantation in a patient, the screw 208 is threaded into the sleeve 110, thereby connecting the linear actuator assembly 200 to the frame 102. The frame 102 can then be placed in a radially collapsed state and the prosthetic valve and the distal end portion of the delivery apparatus can be inserted in a patient. Once the prosthetic valve 100 is at a desired implantation site, the frame 102 can be radially expanded as described herein.

To radially expand the frame 102, the support tube 206 is held firmly against the stopper 112. The actuator member 202 is then pulled in a proximal direction through the support tube 206, such as by pulling on the proximal end of the actuator member 202 or actuating a control knob on the handle that produces proximal movement of the actuator member 202. Because the support tube 206 is being held against the stopper 112, which is connected to the proximal end of the frame 102, the proximal end of the frame 102 is prevented from moving relative to the support tube 206 and the handle. As such, movement of the actuator member 202 in a proximal direction results in movement of the distal end of the frame 102 in a proximal direction causing the frame 102 to foreshorten axially and expand radially.

It should be understood that the frame 102 can also be radially expanded by pushing the proximal end of the frame toward the distal end of the frame by pushing the support tube 206 against the stopper 112 while keeping the actuator member 202 stationary relative to the handle, or alternatively, by simultaneously pushing the support tube 206 distally against the stopper 112 and pulling the actuator member 202 proximally.

After the frame 102 is expanded to a desired radially expanded size, one or more locking mechanisms can be actuated to lock the frame 102 in the desired radially expanded size, and the linear actuator assembly 200 can be disconnected from the frame 102. To disconnect the linear actuator assembly 200 from the frame 102, the actuator member 202 can be rotated so as to unscrew the screw 208 from the stopper 112. The actuator member 202 and the cover tube 204 can then be retracted proximally through the stopper 112 and the linear actuator assembly 200 (including the actuator member 202, the screw 208, the cover tube 204, and the support tube 206) can be withdrawn from the patient. The cover tube 204 facilitates passage of the screw 208 through the stopper 112. In some embodiments, the cover tube 204 can be excluded. In embodiments that have more than one linear actuator assembly 200, the above procedure for expanding the frame 102 is performed for each linear actuator assembly 150.

Further details of the actuator assemblies and various exemplary locking mechanisms can be found in U.S. Publication No. 2018/0153689.

FIG. 5 illustrates a delivery apparatus 300 (also referred to herein as a delivery device), according to one embodiment, adapted to deliver a prosthetic heart valve (e.g., prosthetic valve) 308, such as the prosthetic heart valve 100 illustrated in FIGS. 2-4 and/or the prosthetic valve 10 illustrated in FIG. 1, as described above. The prosthetic valve 308 can be releasably coupled to the delivery apparatus 300, as described further below. It should be understood that the delivery apparatus 300 and other delivery apparatuses disclosed herein can be used to implant prosthetic devices other than prosthetic valves, such as stents or grafts.

The delivery apparatus 300 in the illustrated embodiment generally includes a handle 302, an elongate shaft 304 (which comprises an outer, or outermost, shaft in the illustrated embodiment) extending distally from the handle 302, an inner (e.g., innermost) shaft 310, and at least one actuator assembly (e.g., member or actuator) 306 for expanding and compressing the prosthetic valve extending through the outer shaft 304 and distally outwardly from a distal end portion 312 of the outer shaft 304.

The inner shaft 310 can define an inner lumen that is configured to receive a guidewire therein. For example, during delivery of an implantable medical device (e.g., prosthetic heart valve) to the target implantation site with the delivery apparatus 300, the delivery apparatus 300 can be advanced over the guidewire to the target implantation site.

The delivery apparatus 300 can include three actuator assemblies 306 (only two of the three are shown in FIG. 5) releasably coupled to the prosthetic valve. However, in alternate embodiments, the delivery apparatus 300 may include more or less than three actuator assemblies 306 (e.g., one, two, four, or the like). As shown in FIG. 5, the plurality of actuator assemblies 306 are circumferentially spaced apart from each other around a circumference of the delivery apparatus 300 and can extend axially through the outer shaft 304 from the handle 302 to the prosthetic valve 308.

In particular embodiments, each actuator assembly 306 can be releasably coupled to a corresponding actuator of the prosthetic valve (e.g., a push-pull mechanism 32 as shown in FIG. 1). Each actuator assembly 306 can include an inner member (similar to inner member 42 shown in FIG. 1) having a distal end releasably coupled to an inner member 34 of a push-pull mechanism 32 and an outer member having a distal end releasably coupled to an outer member 36 of a push-pull mechanism 32. In another embodiment, each actuator assembly 306 can be an actuator assembly 200 releasably coupled to the prosthetic valve via a threaded sleeve 110.

As shown in FIG. 5, a distal end of the inner shaft 310 may include a nosecone 314 which may be used to guide the delivery apparatus 300 through a lumen of a patient to a target implantation site for the prosthetic valve 308. The nosecone 314 may be arranged proximate to a distal end of the prosthetic valve 308.

In use, the delivery apparatus 300 can be releasably coupled to the prosthetic valve 308 to produce radial expansion and compression of the frame of the prosthetic valve 308. In some embodiments, the actuator assemblies 306 of the delivery apparatus 300 can be configured to transfer pushing and/or pulling forces from the handle 302 of the delivery apparatus 300 to the prosthetic valve 308. For example, in some embodiments, the actuator assemblies 306 may have distal end portions that can be releasably connected to the prosthetic valve 308 via respective release-and-locking units.

In some embodiments, the outer shaft 304 of the delivery apparatus 300 can be configured as a steerable guide catheter having an adjustable curvature for use in steering the delivery apparatus 300 through the patient's vasculature. In particular embodiments, the outer shaft 304 can include a steerable distal section, the curvature of which can be adjusted by the operator to assist in guiding the apparatus through the patient's vasculature.

The outer shaft 304 and the actuator assemblies 306 can be moved relative to one another (axially and/or rotationally) to facilitate delivery and positioning of the prosthetic valve 308 at an implantation site in the patient's body.

In some embodiments, the distal end portion 312 of the outer shaft 304 can form and/or function as a sheath (e.g., capsule) that is sized and shaped to receive and house the prosthetic valve 308 in a radially compressed state for delivery into and through a patient's vasculature. Once the prosthetic valve 308 is advanced to the implantation site or adjacent the implantation site, the prosthetic valve 308 can be advanced from the sheath by advancing the actuator assemblies 306 relative to the outer shaft 304, after which the prosthetic valve 308 can be radially expanded. In alternative embodiments, the outer shaft 304 can be configured to move axially relative to the actuator assemblies 306 and the prosthetic valve.

The advancement of the prosthetic valve 308 from the sheath by axially moving the actuator assemblies 306 relative to the outer shaft 304 or by retracting the outer shaft 304 relative to the actuator assemblies 306 may be actuated by operating a first knob 316 on the handle 302. The first knob 316 can be operatively connected to a proximal end portion of the outer shaft 304 and can be configured to retract the outer shaft 304 proximally relative to the actuator assemblies 306 to deploy the prosthetic valve 308 from the distal end portion 312 of the sheath or operatively connected to proximal ends of the actuator assemblies 306 to advance the actuator assemblies 306 distally relative to the outer shaft 304 to deploy the prosthetic valve 308 from the distal end portion 312 of the sheath. The first knob 316 may be a slidable or rotatable adjustment element that is operatively connected to the actuator assemblies 306 and/or the outer shaft 304.

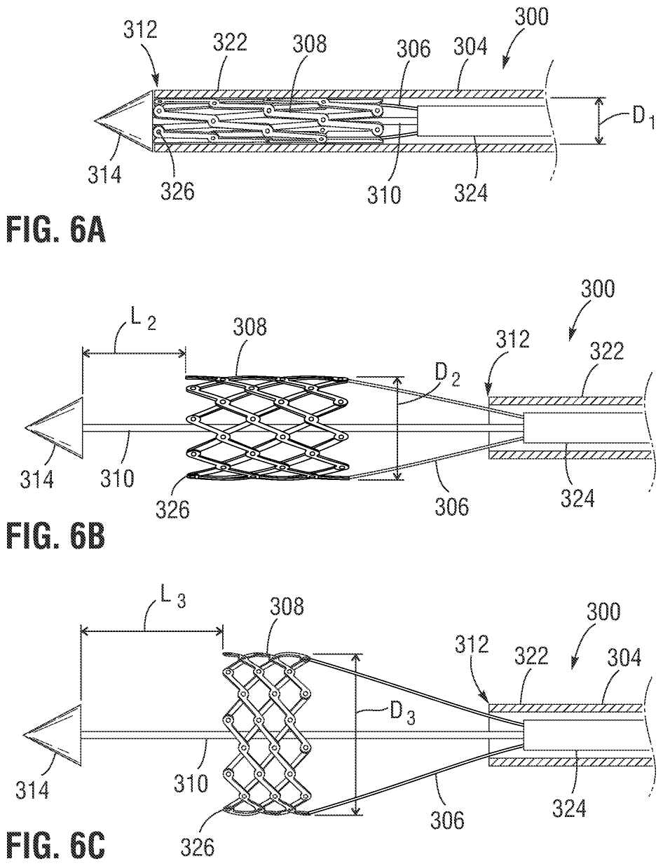

The handle 302 may include additional adjustment knobs, such as a second knob 318 and a third knob 320, as shown in FIG. 5. In some embodiments, the second knob 318 may be operatively coupled to the actuator assemblies 306 and actuate the actuator assemblies 306 to adjust the prosthetic valve 308 from a non-expanded (or radially compressed) configuration (as shown in FIG. 6B, as described below) to a radially expanded configuration (as shown in FIG. 6C, as described below), and vice versa.

In some embodiments, the third knob 320 may be operatively coupled to the actuator assemblies 306 and actuate the actuator assemblies 306 to disconnect from the prosthetic valve 308. As a result, the prosthetic valve 308 may be detached from the delivery apparatus 300 and implanted (e.g., deployed) at the target implantation site.

Turning now to FIGS. 6A-6C, a portion of the delivery apparatus 300 is shown in various stages of a prosthetic valve placement (e.g., implantation) procedure. As described above with reference to FIG. 5, the delivery apparatus 300 includes an outer shaft 304 with a distal end portion 312 that forms a sheath (e.g., capsule) 322 adapted to house the crimped (radially compressed) prosthetic valve 308 during delivery of the prosthetic valve 308 to the target implantation site. The delivery apparatus 300 further includes an inner shaft 310 with a nosecone 314 mounted on a distal end of the inner shaft 310. The inner shaft 310 extends through an interior of the outer shaft 304.

In some embodiments, as shown in FIGS. 6A-6C, the delivery apparatus 300 may also include an intermediate shaft 324 arranged coaxial with and between (in the radial direction relative to a central longitudinal axis of the delivery apparatus) the outer shaft 304 and the inner shaft 310. The intermediate shaft 324 may be adapted to house and organize the actuator assemblies 306. For example, the actuator assemblies 306 may be housed within and extend outwardly from a distal end of the intermediate shaft 324. In some embodiments, each actuator assembly 306 may be kept separate from the other actuator assemblies 306 within the intermediate shaft 324. For example, each actuator assembly 306 can extend through a separate lumen of the intermediate shaft 324.

Though only two actuator assemblies 306 are shown in FIGS. 6A-6C, the delivery apparatus 300 may include three actuator assemblies 306 arranged around a circumference of a frame of the prosthetic valve 308.

FIG. 6A shows the prosthetic valve 308 retained in a radially compressed state within the sheath 322 of the delivery apparatus 300. As such, in FIG. 6A, the prosthetic valve 308 is in its radially compressed configuration having a smallest diameter, D1. The smallest diameter D1 may be approximately the same as an inner diameter of the sheath 322. The sheath 322 surrounding an outside of the prosthetic valve 308, as shown in FIG. 6A, may maintain the prosthetic valve in the radially compressed configuration. As a result, the prosthetic valve 308 may be advanced through a patient's vasculature, for example, to the target implantation site via the delivery apparatus 300.

As shown in FIG. 6A, a distal end 326 of the prosthetic valve 308 is arranged adjacent to a proximal end of the nosecone 314. Thus, there may be little to no gap between the nosecone 314 and the distal end 326 of the prosthetic valve 308.

After reaching the target implantation site, the sheath 322 may be pulled away from the nosecone 314 and the prosthetic valve 308, in a proximal direction along a central longitudinal axis of the delivery apparatus 300, to uncover the prosthetic valve 308. In alternate embodiments, the actuator assemblies 306 may be advanced, in the distal direction, to move the prosthetic valve 308 out of the sheath 322. FIG. 6B shows the prosthetic valve 308 is this uncovered (e.g., unsheathed) state where it is arranged outside of the sheath 322. At this state, the prosthetic valve 308 is not actively expanded via the actuator assemblies 306. However, since it is no longer bound by (e.g., retained within) the sheath 322, the prosthetic valve 308 may assume a partially expanded diameter D2 which is larger than the smallest diameter D1 due to the inherent resiliency of the struts of the frame. For example, after being deployed from the sheath 322, the prosthetic valve 308 may expand, in the radial direction relative to the central longitudinal axis of the valve and delivery apparatus 300, by 10-20%. It should be noted that the extent of expansion of the prosthetic valve 308, from the compressed, smallest diameter D1 (FIG. 6A) to the partially expanded diameter D2 (FIG. 6B) may be exaggerated in FIG. 6B for the purposes of illustration. The expansion in diameter of the prosthetic valve 308 from the smallest diameter D1 to the partially expanded diameter D2 may form a gap having a length of L2 between the distal end 326 of the prosthetic valve 308 and the proximal end of the nosecone 314.

FIG. 6C shows the prosthetic valve 308, after being actively expanded via actuation of the actuator assemblies 306. For example, from FIG. 6B to FIG. 6C, a user may actuate the actuator assemblies 306 (e.g., via the second knob 318 of the handle 302 shown in FIG. 5) to radially expand the prosthetic valve 308. As a result, the prosthetic valve 308 may be radially expanded to an expanded diameter D3, as shown in FIG. 6C. The expanded diameter D3 is larger than the partially expanded diameter D2. As a result of the larger, expanded diameter D3, the gap between the nosecone 314 and the distal end 326 of the prosthetic valve 308 may increase to length L3.

In the partially expanded state, as shown in the example of FIG. 6B, the gap formed between the distal end of the valve and the nosecone may create a discontinuity. If repositioning of the prosthetic valve at the target implantation site is required at this stage, this discontinuity makes it difficult to advance the prosthetic valve in the distal direction, especially if the user is trying to re-cross the native aortic annulus. Further, in some embodiments, it may be necessary to reposition the prosthetic valve, even after partial or full expansion of the prosthetic valve. Repositioning or re-crossing of the prosthetic valve may require at least partial compression of the valve, followed by repositioning (e.g., in a distal or proximal direction), and re-expansion at the new position. A gap between the nosecone and the distal end of the prosthetic valve may make it difficult to reposition the valve. For example, it may be difficult to reposition the valve, in the distal direction and/or the proximal direction, without the valve contacting the patient's anatomy, due to the formed gap.

In some cases, the actuator assemblies 306 can be configured to prevent any expansion of the prosthetic valve 308 after it is advanced from the sheath 322 but before the actuator assemblies are used to actively expand the prosthetic valve. In the other words, the prosthetic valve 308 can have a diameter equal to D1 after it is advanced from the sheath 322. If there is a gap between prosthetic valve 308 and the nosecone 314 when the prosthetic valve is retained in the sheath 322, the gap typically remains after the prosthetic valve is advanced from the sheath 322. In such cases, the gap can make re-crossing the native leaflets difficult.

In some embodiments, a gap between the nosecone and the distal end of the prosthetic valve may form after expansion of a non-mechanical prosthetic valve (e.g., a balloon-expandable or self-expanding prosthetic valve). In some instances, repositioning of these types of valves, after expansion, may be required. However, similarly to as explained above, this gap may make repositioning of these types of prosthetic valves difficult.

Thus, it may be desirable to reduce the gap formation between a fully compressed, or partially or fully expanded prosthetic valve and the nosecone of the delivery apparatus to allow for easier repositioning of the valve without causing injury to a patient's anatomy. As one example, forming a continuity (e.g., a continuous transition) between the nosecone and the distal end of the prosthetic valve, even after partial or full expansion of the valve, may reduce (and in some cases, eliminate) this gap, thereby allowing for easier repositioning of the prosthetic valve at the target implantation site.

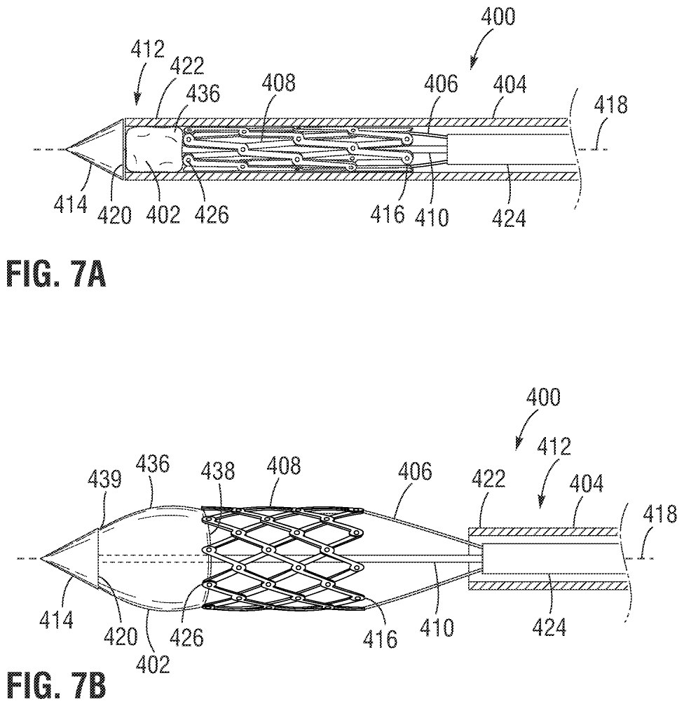

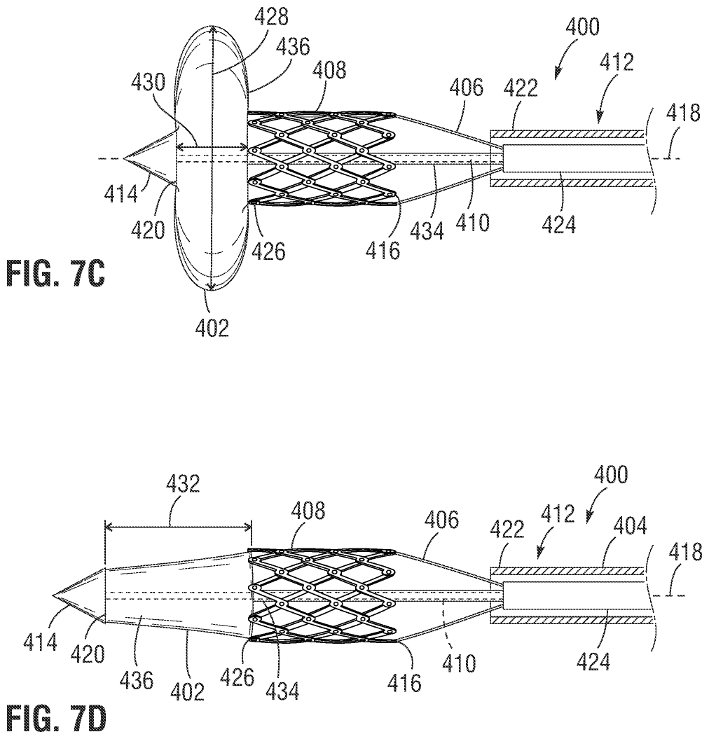

For example, in some embodiments, a delivery device (e.g., apparatus) adapted to deliver a prosthetic medical device, such as a prosthetic heart valve, to a target implantation site, may include a transition element adapted to be positioned between a nosecone of the delivery device and the prosthetic medical device, after being deployed from an interior of a sheath of an outer shaft the delivery device. In some embodiments, as shown in FIGS. 7A-7D, the transition element may be a balloon. In some such embodiments, the balloon may be an inflatable balloon, positioned within the outer shaft in a deflated during a device delivery process and then actively inflated between a distal end of the device and a proximal end of the nosecone, after deploying the device from the sheath (in case re-crossing or repositioning is required; otherwise the balloon need not be inflated). In other such embodiments, the balloon may be pre-filled (e.g., pre-inflated) and compressed within the outer shaft (or within another tube or shaft of the delivery apparatus) during the delivery process and then passively expanded between the nosecone and the device after deploying the device from the sheath.

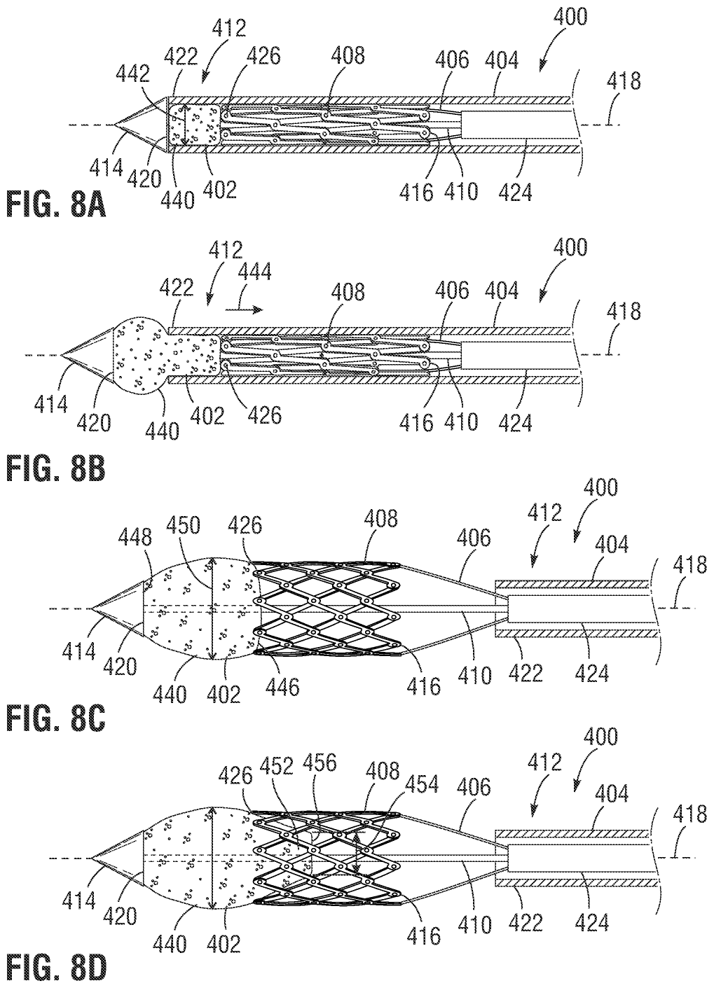

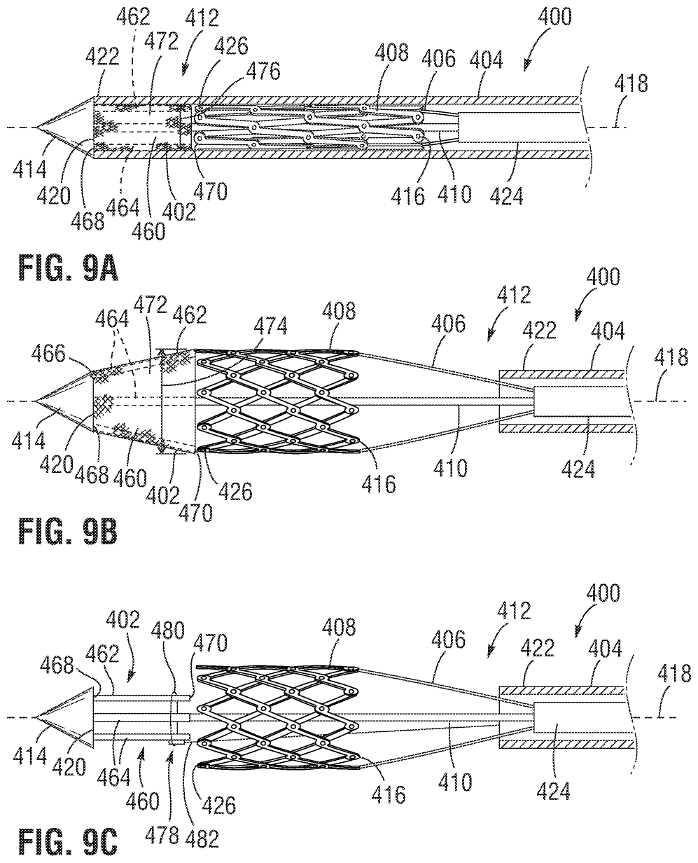

In other embodiments, as shown in FIGS. 8A-8C, the transition element may be a compressible element, such as a sponge. In yet other embodiments, as shown in FIGS. 9A-9C, the transition element may be a mechanical element comprising an expandable frame. In this way, the transition element may form a continuous transition between the nosecone and the prosthetic medical device, after deployment from the sheath.

FIGS. 7A-9C show embodiments of a delivery device (e.g., apparatus) 400 including a transition element 402 adapted to be positioned between a nosecone 414 of the delivery device 400 and a partially expanded prosthetic valve (e.g., prosthetic heart valve) 408, after deployment from a sheath 422 of the delivery device 400. These embodiments also can be used in cases where a gap exists between a fully compressed prosthetic valve and a nosecone 414 after deployment from a sheath 422 (i.e., in cases where the prosthetic valve has a diameter D1 after deployment from the sheath). Similar to the delivery apparatus 300 described above with reference to FIGS. 5 and 6A-6C, the delivery device 400 includes an outer shaft 404 which may extend distally from a handle (not shown in FIGS. 7A-9C) of the delivery device 400. The outer shaft 404 has a distal end portion 412 that forms a sheath (e.g., capsule) 422 adapted to house the prosthetic valve 408 in a radially compressed (e.g., crimped) configuration during delivery of the prosthetic valve 408 to the target implantation site.

The delivery device 400 further includes an inner shaft 410 with a nosecone 414 mounted on a distal end of the inner shaft 410. The inner shaft 410 extends through an interior of the outer shaft 404.

In some embodiments, the delivery device 400 may also include an intermediate shaft 424 arranged coaxial with and between the outer shaft 404 and the inner shaft 410. The intermediate shaft 424 may be adapted to house and organize one or more actuator assemblies (e.g., actuators) 406. For example, the actuator assemblies 406 may be housed within and extend outwardly from a distal end of the intermediate shaft 424.

The prosthetic valve 408 includes a frame with a proximal end 416 and a distal end 426, the distal end 426 arranged opposite the proximal end 416, in a direction of a central longitudinal axis 418 of the delivery device 400 (and valve). The actuator assemblies 406 may be coupled to the proximal end 416 of the frame of the prosthetic valve 408. The distal end 426 of the frame of the prosthetic valve 408 is arranged proximate to a proximal end 420 of the nosecone (e.g., the proximal end 420 is arranged closer to the distal end 426 than the proximal end 416 of the frame of the prosthetic valve 408).

While the prosthetic valve 408 illustrated in FIGS. 7A-9C is a mechanically expandable valve, in alternate embodiments, the prosthetic valve 408 may be a balloon expandable or self-expandable valve. As such, the delivery device 400 may not include the actuator assemblies 406 and may instead include an inflatable balloon, sheath, or no additional component for expanding the prosthetic valve 408 if the prosthetic valve is fully self-expandable.

In some embodiments, as shown in FIGS. 7A-7D, the transition element 402 is a balloon 436. As shown in FIG. 7A, the prosthetic valve 408 is retained in a radially compressed (e.g., crimped) state within the sheath 422 of the outer shaft 404. The balloon 436 is also arranged within the outer shaft 404. In some embodiments, as shown in FIG. 7A, the balloon 436 may be arranged within the sheath 422, between the distal end 426 of the prosthetic valve 408 and the proximal end 420 of the nosecone 414 (in an axial direction relative to the central longitudinal axis 418). In alternate embodiments, the balloon 436 may be arranged within the outer shaft 404, in an alternate location (e.g., such as proximal to the prosthetic valve 408). In these embodiments, the prosthetic valve 408 may be arranged adjacent to the nosecone 414 and after retraction of the sheath 422, the balloon 436 may be advanced in the proximal direction, through an interior of the no longer compressed prosthetic valve 408, and into a gap formed between the prosthetic valve 408 and the nosecone 414.

In some embodiments, the balloon 436 is an inflatable balloon adapted to be inflated from a deflated state (as shown in FIG. 7A) to an inflated state (as shown in FIG. 7B, as described further below). For example, the balloon 436 may be retained in a deflated (e.g., non-inflated) state, within the sheath 422, when the prosthetic valve 408 is also retained within the sheath 422 (in the radially compressed state), as shown in FIG. 7A. After the prosthetic valve 408 is deployed from the sheath 422 (e.g., via retracting the outer shaft 404 axially, in a proximal direction, and/or advancing the valve 408 axially out of the outer shaft 404, in a distal direction) and becomes uncovered (e.g., not encased by the sheath 422), the prosthetic valve 408 may assume a partially expanded state (e.g., not actively expanded by the actuator assemblies), having a partially expanded diameter (as described above with reference to FIG. 6B) that is larger than its radially compressed diameter when arranged inside the sheath 422. The balloon 436 may then be actively inflated, as shown in FIG. 7B, via an inflation device.

In one embodiment, a balloon catheter may be used to inflate and deflate the balloon 436. For example, a balloon catheter may extend through the intermediate shaft 424 and/or inner shaft 410 and fluidly couple to the balloon 436. In another embodiment, an inner lumen of the inner shaft 410 may be used to deliver an inflation fluid (e.g., saline) to the balloon 436 via one or more ports or openings arranged along the inner shaft 410, in a region of the inner shaft 410 that is arranged inside the balloon 436.

As shown in FIG. 7B, the balloon 436 is inflated to a larger, outer diameter that forms an outer surface that creates a continuous transition between the distal end 426 of the prosthetic valve 408 and the proximal end 420 of the nosecone 414. For example, the outer surface of the balloon 436 may form a curved surface that curves and/or tapers from the distal end 426 and the proximal end 420. As shown in FIG. 7B, in the expanded state, a proximal end 438 of the balloon 436 contacts the distal end 426 of the prosthetic valve 408 and a distal end 439 of the balloon 436 contacts the proximal end 420 of the nosecone 414. In some embodiments, the distal end of the balloon 436 can be affixed to the proximal end 420 of the nosecone 414 or can be integrally formed with the nosecone.

The balloon 436 may be inflated by an amount that provides this continuous transition between the proximal end 420 of the nosecone 414 and the distal end 426 of the prosthetic valve 408.

In some embodiments, the balloon 436 can be a compliant balloon formed from an elastic material (e.g., polyurethane or silicone). A compliant balloon 436 can be inflated to a desired size within a range of possible sizes based on the size of the prosthetic valve 408. In other embodiments, the balloon 436 can be a semi-compliant balloon formed from a material that is relatively less elastic than materials used for compliant balloons (e.g., Pebax or high-durometer polyurethanes). Similar to a compliant balloon, a semi-compliant balloon can be inflated to a desired size within a range of possible sizes based on the size of the prosthetic valve 408, although it cannot stretch or expand to the extent that a compliant balloon can.

In still other embodiments, the balloon 436 can be a noncompliant balloon formed from a non-elastic material or material with a small amount of elasticity (e.g., polyester or nylon). A noncompliant balloon expands to a predetermined size when fully inflated, which can be selected based on the size of the prosthetic valve with which the balloon will be used.

The inflated balloon 436, as shown in FIG. 7B, enables easier repositioning of the prosthetic valve 408, particularly in a distal direction, if such repositioning is required after reaching the target implantation site. For example, the continuous transition between the nosecone 414 and the prosthetic valve 408 provided by the inflated balloon 436 may increase the maneuverability of the prosthetic valve 408 via the delivery device 400 without the prosthetic valve coming into contact with and/or inuring the patient's anatomy at the target implantation site.

Once the prosthetic valve 408 is actively expanded (as shown in FIG. 6C, for example) and implanted at the target implantation site, the balloon 436 may be deflated, for example to the deflated state shown in FIG. 7A, and then retracted through an inner lumen of the expanded prosthetic valve 408 back into the sheath 422. In this way, the balloon 436 may be deflated to reduce its diameter for easier removal from the target implantation site and through the patient's vasculature, without displacing the implanted valve.

In some embodiments, the distal end of the balloon 436 may be attached to the proximal end 420 of the nosecone 414.

In other embodiments, as shown in FIGS. 7C and 7D, the balloon 436 may be a pre-inflated (or pre-filled) balloon, which can be actively expanded or adapted to passively expand from a compressed state (as shown in FIG. 7A) to an expanded state (as shown in FIGS. 7C-7D), as described further below.

As an example, the balloon 436 may be pre-filled with a compressible fluid or other type of compressible material (such as with a hydrogel, which can be in the form of hydrogel beads) to an expanded state and then compressed (to a smaller diameter) to fit within the sheath 422, between the nosecone 414 and the prosthetic valve 408, as shown in FIG. 7A. Then, when the sheath 422 is retracted away from the prosthetic valve 408, to uncover and deploy the valve, the pre-filled balloon 436 may passively expand (the amount of expansion based on its pre-filled size or diameter). For example, the pre-filled (e.g., pre-inflated) balloon may assume its pre-inflated size (e.g., diameter) when in the (radially) expanded state.

In some embodiments, the shape of the balloon (whether pre-filled or inflated by the user) can be modified by the user by moving the proximal and distal ends of the balloon relative to each other. For example, as shown in FIGS. 7C and 7D, the pre-filled balloon may be attached at its proximal end (e.g., the end closest to the prosthetic valve 408) to a pull member 434, such as a cable or shaft, and at its distal end to the nosecone 414 and/or the inner shaft 410. The pull member 434 may be configured to apply a pull force (e.g., axially in the proximal direction) or a push force (e.g., axially in the distal direction) on the proximal end of the balloon 436. Moving the pull member 434 axially relative to the inner shaft 410, and vice versa, is effective to adjust the length and the diameter of the balloon 436. In particular, retracting the pull member 434 proximally and/or advancing the inner shaft 410 distally is effective to increase the length of the balloon 436 and decrease its diameter (FIG. 7D) (effectively radially compressing the balloon), while advancing the pull member 434 distally and/or retracting the inner shaft 410 proximally is effective to decrease the length of the balloon 436 and increase its diameter (FIG. 7C) (effectively radially expanding the balloon). The balloon can be brought back into the sheath 422 at the end of a procedure by retracting both the inner shaft 410 and the pull member 434 proximally relative to the sheath 422.

In some cases, as shown in FIG. 7C, the balloon 436 may assume an expanded diameter 428 that is larger than a desired outer diameter of the balloon 436. This may occur when the proximal end 420 of the nosecone 414 and the distal end 426 of the prosthetic valve 408 are too close to one another, as shown by the first length 430 which represents a length (in the axial direction) of the balloon 436. In these cases, it may be possible to extend the length of the balloon 436 from the first length 430 (shown in FIG. 7C) to a longer, second length 432, as shown in FIG. 7D.

Extending the length of the balloon 436 to the second length 432 decreases the outer diameter of the balloon 436. In one example, the second length 432 may be chosen so that the largest diameter of the balloon 436 is equal to or slightly less than the outer diameter (e.g., non-actively expanded diameter) of the prosthetic valve 408, as shown in FIG. 7D. In this way, the outer surface of the balloon 436 creates a continuous (and gradual) transition between the outer diameter of the prosthetic valve 408 and the outer diameter of the proximal end 420 of the nosecone 414.

The dimensions, including the length and filled volume of the balloon may be selected to provide a continuous transition between the proximal end 420 of the nosecone 414 and the distal end 426 of the prosthetic valve 408.

Further, in the embodiments of the pre-filled (non-actively inflatable) balloon 436, the length and filled volume of the balloon may be further chosen to enable retraction of the balloon through the inner lumen of the prosthetic valve 408, at the end of the implantation procedure (e.g., after the valve has been actively expanded and placed in the patient's anatomy).

In another implementation, the balloon 436 can be pre-filled with a liquid (e.g., saline). The balloon can be radially compressed by retracting the pull member 434 proximally and/or advancing the inner shaft 410 distally to reduce the diameter of the balloon 436 until it is equal to or less than D1 and can be stored in the sheath 422 during delivery of the prosthetic valve. At the implantation site, the prosthetic valve 408 and the balloon 436 can be deployed from the sheath 422. The user can then adjust the size of the balloon 436 to create a smooth transition section between the prosthetic valve and the balloon, as depicted in FIG. 7C.

The pre-filled balloon does not require an inflation/deflation catheter, which may simplify the overall structure of the delivery device 400. In alternative embodiments, the balloon can be pre-filled but can also be configured to receive additional inflation fluid during the implantation procedure to further increase the size of the balloon if needed.

In an alternative embodiment, the configuration shown in FIGS. 7C and 7D can be used to adjust the length of an inflatable balloon, either prior to or after inflating the balloon with an inflation medium.

In this way, a balloon (actively inflatable or pre-inflated) of a delivery device may be adapted to be positioned between a nosecone and prosthetic valve, after unsheathing the prosthetic valve from an outer shaft of the delivery device, thereby providing a continuous transition and filling a gap created between the nosecone and the non-compressed prosthetic valve. As a result, the prosthetic valve may be more easily repositioned at the target implantation site, if required, without causing damage to the patient's anatomy and/or the prosthetic valve.

In some embodiments, as shown in FIGS. 8A-8C, the transition element 402 is a compressible element, such as a compressible foam or sponge, 440. For example, in some embodiments, the compressible element 440 may comprise a foam or sponge material that is compressible, relatively soft, and/or porous. As an example, the compressible element 440 may comprise a compressible material, such as foam or sponge, that allows it to be compressed upon application of a compression force and then return (e.g., spring back) to its resting or non-compressed size after the compression force is removed.

Thus, the compressible element 440 may have an expanded, non-compressed (e.g., resting) state or geometry when not retained within and compressed by the sheath 422 of the delivery device 400 (as shown in FIGS. 8C and 8D). Further, the compressible element 440 may be compressible, into a radially compressed state or geometry (having a smaller outer diameter than the expanded, non-compressed geometry).

For example, as shown in FIG. 8A, the compressible element 440 is retained in a (radially) compressed state, having a first diameter 442, within the sheath 422 of the delivery device 400. The compressible element 440 is arranged within the sheath 422, in a space between, in a direction of the central longitudinal axis 418, the proximal end 420 of the nosecone 414 and the distal end 426 of the prosthetic valve 408. In this way, the compressible element 440 may be arranged directly adjacent to each of the nosecone 414 and the prosthetic valve 408. The inner shaft 410 can extend through the compressible element 440. The compressible element 440 may be affixed to the nosecone 414 and/or the inner shaft 410.

The compressible element 440 is configured to expand to its resting (e.g., expanded, non-compressed) state, between the nosecone 414 and the prosthetic valve 408 when the sheath 422 is moved away from and no longer covers the compressible element 440 and the prosthetic valve 408.

For example, as shown in FIG. 8B, when the sheath 422 is partially pulled away from the nosecone 414, in the proximal direction 444, a distal portion (e.g., the portion arranged adjacent to the nosecone 414) of the compressible element 440 is uncovered and exposed to the exterior environment (outside the sheath 422). As a result, the distal portion of the compressible element 440, which is no longer arranged within the interior of the sheath 422, may expand to a diameter that is greater than the first diameter 442. However, the portion (e.g., proximal portion) which remains enclosed within the sheath 422 retains its compressed, first diameter 442.

In FIG. 8C, the sheath 422 is pulled back, in the proximal direction 444, even further to expose and uncover the entire compressible element 440 and the prosthetic valve 408. As a result, the prosthetic valve 408 expands to a partially expanded state, which in some embodiments, may also be a non-actively expanded state. Thus, a diameter of the prosthetic valve 408 may be larger in its non-actively expanded state than its radially compressed diameter, as shown in FIG. 8A.

After being fully deployed from the sheath 422 (e.g., arranged outside of the sheath), the compressible element 440 expands to its resting state (also referred to as its expanded, non-compressed state) having a second diameter 450, as shown in FIG. 8C. The second diameter 450 is larger than the first diameter 442. In the expanded state, a proximal end 446 of the compressible element 440 can contact the distal end 426 of the prosthetic valve 408 and a distal end 448 of the compressible element 440 can contact the proximal end 420 of the nosecone 414.

In this way, due to its compressible nature, the compressible element 440 is adapted to passively expand (e.g., without active actuation from an external, actuation source) from its compressed state to its expanded, non-compressed state upon removal from an inside of the sheath 422. This is due to the face that inner walls of the sheath 422 are no longer applying an inward, compression force against an outer surface of the compressible element 440.

As shown in FIG. 8C, the outer surface of the compressible element 440 creates a continuous transition from the distal end 426 of the prosthetic valve 408 to the proximal end 420 of the nosecone 414. For example, the outer surface of the compressible element 440 may form a curved surface that curves between the distal end 426 and the proximal end 420.

For example, in some embodiments, the compressible element 440 tapers in diameter from the second diameter 450, at a middle portion of the compressible element 440, to the proximal end 420 of the nosecone 414 and tapers in diameter from the second diameter 450, at the middle portion, to the distal end 426 of the prosthetic valve 408.

In some embodiments, as shown in FIG. 8D, the compressible element 440 has a proximal tapered region 452 that tapers to a third diameter 454 at a proximal-most end 456 of the compressible element 440. The third diameter 454 is smaller than a diameter of the prosthetic valve 408 (in its non-compressed state, as shown in FIG. 8D) and smaller than the second diameter 450.

In some embodiments, as shown in FIG. 8D, the proximal tapered region 452 is arranged within an interior of the prosthetic valve 408 and extends partway into the interior of the prosthetic valve 408, from the distal end 426 of the prosthetic valve 408. This tapering allows the distal end of the prosthetic valve to partially overlap the compressible element 440 to ensure there is a smooth transition between the prosthetic valve 408 and the compressible element 440. Further, this tapering enables the compressible element 440 to be compressed against either the at least partially expanded frame of the prosthetic valve 408 or the distal lip of the sheath 422, to enable easy retraction (in the proximal direction 444) at the end of the valve implantation procedure. Thus, in some embodiments, the compressible element 440 having the proximal tapered region 452, may be more easily retracted through the prosthetic valve 408 and removed from the implantation site and the patient.

In some embodiments, the proximal end 446 (or the proximal-most end 456 in embodiments where the compressible element has the proximal tapered region 452) of the compressible element 440 may be attached to a pull member (not shown in FIGS. 8A-8D) (in lieu of or in addition to being attached to the inner shaft 410 or the nosecone 414), such as a cable or shaft, configured to apply a pull force in the proximal direction 444 for retraction of the compressible element 440 to move it closer to the prosthetic valve 408 or for retraction away from the implantation site, at the end of the procedure.

Due to its compressible nature, the compressible element 440 may compress to a smaller diameter (e.g., smaller than second diameter 450) during removal from the implantation site, at the end of the implantation procedure, and may not disrupt or dislodge the radially expanded and implanted prosthetic valve 408.

In this way, a compressible element (e.g., compressible foam or sponge) of a delivery device may be adapted to be positioned between a nosecone and prosthetic valve, after unsheathing the prosthetic valve from an outer shaft of the delivery device, thereby providing a continuous transition between the nosecone and the partially expanded prosthetic valve. As a result, the prosthetic valve may be more easily repositioned at the target implantation site, if required, without causing damage to the patient's anatomy and/or the prosthetic valve.

In some embodiments, as shown in FIGS. 9A-9C, the transition element 402 is an expandable, mechanical element 460 comprising an expandable frame 462. The mechanical element 460 is moveable between a radially compressed state (as shown in FIG. 9A) to an expanded state (as shown in FIG. 9B). In its expanded state, the mechanical element 460 is configured to provide a continuous transition, in the axial direction, between the nosecone 414 and the frame of the prosthetic valve 408.

As shown in FIGS. 9A-9C, the expandable frame 462 can comprise a plurality of arms 464 attached to a proximal region of the nosecone 414. In some embodiments, a distal end 468 of each of the arms 464 may be coupled to the proximal end 420 of the nosecone 414.

In some embodiments, the distal end 468 of each of the arms 464 may be coupled to the proximal end 420 of the nosecone 414 via a hinged connection 466. As such, each arm 464 may be configured to pivot about its hinged connection 466 between a compressed state (as shown in FIG. 9A) and an expanded state (as shown in FIG. 9B).

Each arm 464 extends proximally, in the axial direction, towards the prosthetic valve 408, from its distal end 468 to a proximal end 470 of the arm 464. The proximal end 470 of each arm 464 may be a free end that is unattached to another component of the delivery device 400, and thus, is adapted to freely move from the compressed state to the expanded state.

In some embodiments, the arms 464 can be covered by a circumferential flexible cover 472 (shown in FIGS. 9A-9B). The cover 472 may comprise a fabric (e.g., cloth), flexible polymer, and/or the like. For example, the cover 472 may overlap and cover an outer surface of each of the arms 464 and surround, around a circumference of the mechanical element 460, the frame 462. In this way, the mechanical element 460 may form a sleeve including the mechanical, expandable frame 462 and the cover 472.

As shown in FIG. 9A, the frame 462 can be retained in its radially compressed state within the sheath 422, when the sheath 422 encloses both the prosthetic valve 408 and the frame mechanical element 460, with its arms 464 spring-biased against the inner wall of the sheath 422. For example, the frame 463 may be retained in its radially compressed state via inward compression forces from the surrounding inner walls of the sheath 422.

Then, when the sheath 422 is removed to uncover the frame 462 (e.g., retracted in the proximal direction, away from the nosecone 414), the frame 462 assumes its expanded configuration, tapering in diameter from the prosthetic valve 408 to the nosecone 414.

For example, as shown in FIG. 9B, upon moving the sheath 422 axially away from the frame 462 to uncover the frame 462 and the prosthetic valve 408, the proximal end 470 of each of the arms 464 may be forced radially outwards (relative to the central longitudinal axis) due to a preloaded spring force. The distal end 468 of each of the arms 464 remains fixed to the nosecone 414, but each arm may pivot about its corresponding hinged connection 466 to the nosecone 414, to allow the proximal end 470 of each arm 464 to expand radially outward to an expanded diameter 474 (shown in FIG. 9B) which is larger than a compressed diameter 476 (shown in FIG. 9A) of the frame 462.

As shown in FIG. 9B, in its expanded state, the frame 462 tapers towards the proximal end 420 of the nosecone 414. For example, in some embodiments, in the expanded state, the proximal end 470 of each arm 464 of the frame 462 contacts the distal end 426 of the prosthetic valve 408 and the distal end 468 of each arm 464 of the frame 462 contacts the proximal end 420 of the nosecone 414.

In this way, the mechanical element 460 extends between and forms a continuous transition between the nosecone 414 and the prosthetic valve 408, after the prosthetic valve 408 has been deployed from within the sheath 422 and assumes an at least partially expanded configuration (as shown in FIG. 9B). Further, the mechanical element 460 fills a gap that may otherwise be created between the at least partially expanded prosthetic valve 408 and the nosecone 414, as explained above with reference to FIGS. 6B-6C.

In some embodiments, as shown in FIG. 9C, the mechanical element 460 can further include a compression mechanism 478 configured to re-compress the frame 462 to its compressed state in order to facilitate the retraction of the frame 462 from the implantation site, through an inner lumen of the expanded prosthetic valve 408 and into the sheath 422, once the implantation procedure is complete. As a result, the mechanical element 460 and nosecone 414 may be retracted, in the proximal direction, away from the implantation site and through the inner lumen of the prosthetic valve, without disrupting or dislodging the implanted prosthetic valve.

It should be noted that FIG. 9C shows the mechanical element 460 without the cover 472 surrounding the frame 462 for illustration purposes. The mechanical element 460 may or may not include the cover 472, in different embodiments. In embodiments where the mechanical element 460 includes the cover 472, the compression mechanism 478 may be adapted to surround the cover 472 and compress the cover 472 and frame 462 together into the compressed state.

As shown in FIG. 9C, in some embodiments, the compression mechanism 478 comprises an adjustable loop 480 (e.g., a wire or suture loop) that wraps around or encircles the arms 464 of the frame 462 and an actuation member 482 (e.g., a wire or suture) configured to reduce the size of the loop and compress the frame 462. Pulling the actuation member 482 proximally (at the handle of the delivery apparatus) is effective to reduce the diameter of the loop, which in turn radially compresses the mechanical element 460). Further details of such compression mechanisms can be found in U.S. Patent Application 62/799,678, incorporated herein by reference in its entirety.

In this way, an expandable mechanical element of a delivery device may be adapted to be positioned between a nosecone and prosthetic valve, after unsheathing the prosthetic valve from an outer shaft of the delivery device, thereby providing a continuous transition between the nosecone and the non-compressed prosthetic valve. As a result, the prosthetic valve may be more easily repositioned at the target implantation site, if required, without causing damage to the patient's anatomy and/or the prosthetic valve.

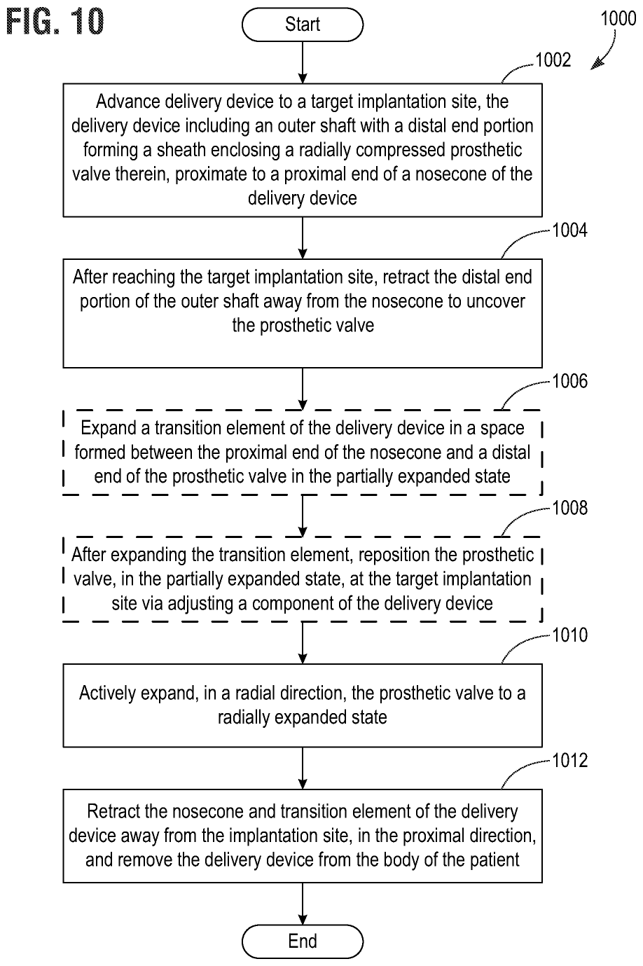

FIG. 10 show a method 1000 for delivering a prosthetic valve to a target implantation site, according to an embodiment. The prosthetic valve may be one of the prosthetic valves described herein, such as prosthetic valve 10 of FIG. 1, prosthetic valve 100 of FIGS. 2-4, prosthetic valve 308 of FIGS. 5-6C, and prosthetic valve 408 of FIGS. 7A-9C.

At 1002, method 1000 includes advancing a delivery device (e.g., delivery device 300 of FIGS. 5-6C and/or delivery device 400 of FIGS. 7A-9C) of a transcatheter delivery system to a target implantation site in a patient (e.g., a heart), the delivery device including an outer shaft with a distal end portion forming a sheath enclosing a radially compressed prosthetic valve therein, proximate to a proximal end of a nosecone of the delivery device. An example of a sheath of an outer shaft of a delivery device enclosing a radially compressed prosthetic valve is shown in FIGS. 6A, 7A, 8A, and 9A, as described above.

At 1004, method 1000 includes, after reaching the target implantation site, retracting (or moving, such as axially moving) the distal end portion of the outer shaft away from the nosecone to uncover the prosthetic valve, which can cause the prosthetic valve to expand to a partially expanded state (e.g., as shown in FIGS. 6B, 7B-7D, 8C-8D, and 9B). For example, when the sheath is moved away, in an axial direction, from the prosthetic valve, the prosthetic valve may expand (e.g., passively, without an active actuation force from an external mechanism) to a partially expanded state, as described above with reference to FIG. 6B. In other cases, the prosthetic valve can remain in a fully compressed state once removed from the sheath.

At 1006, if required for repositioning (e.g., re-crossing the native valve), the method 1000 includes expanding a transition element of the delivery device in a space formed between the proximal end of the nosecone and a distal end of the prosthetic valve in the partially expanded or fully compressed state. The transition element may include one of the transition elements described herein with reference to FIGS. 7A-9C. For example, in some embodiments, the transition element is an inflatable balloon and expanding the transition element includes inflating the inflatable balloon from a deflated state to an inflated state (as shown in FIGS. 7A-7B), between the nosecone and the partially expanded (e.g., non-compressed) or fully compressed prosthetic valve.

In other embodiments, the transition element is a pre-inflated balloon and expanding the transition element includes passively expanding the pre-inflated balloon from a radially compressed state (as shown in FIG. 7A) to a radially expanded state (as shown in FIGS. 7C-7D), between the nosecone and the prosthetic valve, where the pre-inflated balloon assumes its pre-inflated size when in the radially expanded state. Alternatively, the pre-filled balloon can be actively expanded changing its shape from a radially compressed state to a radially expanded state.

In yet other embodiments, the transition element is a compressible element including one of a compressible foam and a sponge material and expanding the transition element includes passively expanding the compressible element from a compressed state (as shown in FIG. 8A) to an expanded, non-compressed state (as shown in FIGS. 8C and 8D), where the compressible element is in its resting state when in the expanded state. In other embodiments, the transition element is a mechanical element comprising an expandable frame having a distal end coupled to the nosecone and expanding the transition element includes expanding a proximal end of the expandable frame from a compressed state (as shown in FIG. 9A) to an expanded state (as shown in FIG. 9B). If needed, the position of the prosthetic valve can be adjusted to place the distal end of the prosthetic valve in contact with or partially overlapping the proximal end of the transition element.

At 1008, method 1000 optionally includes (e.g., if required by the procedure due to inaccurate positioning), after expanding the transition element, repositioning the prosthetic valve, in the partially expanded state or fully compressed state, at the target implantation site via adjusting a component of the delivery device. The more continuous transition provided by the transition element, between the nosecone of the delivery device and the prosthetic valve, may enable easier maneuvering of the valve in the distal or proximal directions during repositioning, without causing degradation to the patient's anatomy and/or the prosthetic valve.

At 1010, method 1000 includes, after repositioning the prosthetic valve, or after positioning the prosthetic valve (without repositioning), actively expanding, in a radial direction, the prosthetic valve to a radially expanded state. For example, actively expanding the prosthetic valve may include actuating one or more actuator assemblies (e.g., actuator assemblies 306 shown in FIGS. 6A-6C and/or actuator assemblies 406 shown in FIGS. 7A-9C) of the delivery device to actively expand the prosthetic valve to its expanded diameter (e.g., D3 shown in FIG. 6C). In alternate embodiments, actively expanding the prosthetic valve may include filling an inflatable balloon of a balloon catheter, around which the prosthetic valve is mounted, to radially expand the prosthetic valve.

At 1012, method 1000 includes retracting the nosecone and transition element of the delivery device away from the implantation site, in the proximal direction, and removing the delivery device from the body of the patient. In some embodiments, the method at 1012 may include compressing the transition element to a geometry (e.g., diameter) that is smaller than its diameter in the expanded state. For example, if the transition element is an inflatable balloon, the method at 1012 may include deflating the balloon and then retracting the nosecone and balloon, in the proximal direction, through an inner lumen of the prosthetic valve. In another example, if the transition element is a compressible element (such as a compressible foam or sponge), the method at 1012 may include pulling the nosecone and compressible element in the proximal through the inner lumen of the prosthetic valve and passively compressible the compressible element to a radially smaller state (e.g., via pressure against the inner lumen of the prosthetic valve). In yet another example, if the transition element is a mechanical element with an expandable (and compressible) frame, the method at 1012 may include re-compressing the mechanical element to its compressed state via a compression mechanism (as shown in FIG. 9C, for example) and then pulling the nosecone and compressed mechanical element, in the proximal direction, through the inner lumen of the prosthetic valve.

In this way, the more continuous transition between an at least partially expanded or fully compressed prosthetic valve (e.g., after being removed from a sheath of a delivery device) and a nosecone of the delivery device provided by one of the transition elements described herein may allow for easier repositioning of the prosthetic valve at or proximate to the target implantation site within a body of a patient. For example, an at least partially expanded or fully compressed prosthetic valve may be more easily moved in a distal and/or proximal direction, relative to a target implantation site, to reposition the prosthetic valve before fully expanding and implanting the prosthetic valve at the target implantation site, without causing damage to the body of the patient and/or the prosthetic valve, when the transition element is utilized. Further, by having a compressible or actively expandable and compressible transition element, the transition element may be stored in a compressed state within an interior of an outer shaft of the delivery device during maneuvering of the delivery device to the target implantation site and then expanded to its expanded, non-compressed state after uncovering of the prosthetic valve from the distal end of the outer shaft, thereby forming the more continuous transition in a space formed between the uncovered prosthetic valve and the nosecone. The compressible transition element may then be re-compressed, prior to removal of the delivery device from the implantation site, through the inner lumen of the expanded prosthetic valve, thereby enabling easier removal that does not disturb or dislodge the implanted prosthetic valve.

### General Considerations

It should be understood that the disclosed embodiments can be adapted to deliver and implant prosthetic devices in any of the native annuluses of the heart (e.g., the pulmonary, mitral, and tricuspid annuluses), and can be used with any of various delivery approaches (e.g., retrograde, antegrade, transseptal, transventricular, transatrial, etc.).

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved. The technologies from any example can be combined with the technologies described in any one or more of the other examples. In view of the many possible embodiments to which the principles of the disclosed technology may be applied, it should be recognized that the illustrated embodiments are only preferred examples and should not be taken as limiting the scope of the disclosed technology.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein, with reference to the transcatheter delivery system, the prosthetic heart valve, the delivery device, the delivery apparatus, and the transition element, “proximal” refers to a position, direction, or portion of a component that is closer to a handle of the delivery system that is outside the patient, while “distal” refers to a position, direction, or portion of a component that is further away from the handle (and farther into a body of the patient). The terms “longitudinal” and “axial” refer to an axis extending in the proximal and distal directions, unless otherwise expressly defined.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and or.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An assembly, comprising
a prosthetic valve; and
a delivery apparatus, comprising:
an outer shaft with a distal end portion forming a sheath adapted to enclose the prosthetic valve therein in a radially compressed configuration;
an inner shaft arranged within the outer shaft and including a nosecone arranged at a distal end of the inner shaft, the nosecone arranged outside of the outer shaft, at the distal end portion of the outer shaft; and
an expandable transition element adapted to expand from a non-expanded state within the outer shaft to an expanded state outside the outer shaft, wherein, in the expanded state, the transition element forms a continuous transition from the nosecone to the prosthetic valve when the sheath is moved away from the nosecone to uncover the prosthetic valve.

2. The assembly of claim 1, wherein the delivery apparatus further comprises at least one actuator assembly arranged within the outer shaft and releasably coupled to the prosthetic valve.

3. The assembly of claim 1, wherein the transition element is a balloon.

4. The assembly of claim 3, wherein the balloon is an inflatable balloon that is inflatable from a deflated state prior to removal of the prosthetic valve from the sheath to an inflated state after removal of the prosthetic valve from the sheath, wherein, when the balloon is in the deflated state, it is arranged within an interior of the sheath, between the nosecone and a distal end of the prosthetic valve, in the radially compressed configuration, and wherein, when the balloon is in the inflated state, it is arranged exterior to the outer shaft and between the nosecone and a distal end of the prosthetic valve.

5. The assembly of claim 3, wherein the balloon is a pre-inflated balloon, pre-inflated to an expanded state, that passively transitions between a compressed state when positioned within the sheath to the expanded state when the sheath is moved away from the balloon.

6. The assembly of claim 1, wherein the transition element is a compressible element including one or more of a compressible foam and a sponge.

7. The assembly of claim 1, wherein the transition element is an expandable, mechanical element.

8. The assembly of claim 7, wherein the mechanical element comprises an expandable frame including a plurality of arms, wherein each arm of the plurality of arms includes a distal end attached to the nosecone and a proximal end that is unattached to the delivery apparatus and adapted to expand from a compressed state to an expanded state, and wherein the mechanical element further comprises a cover surrounding the plurality of arms, around a circumference of the expandable frame.

9. The assembly of claim 1, wherein, in the expanded state, a proximal end of the transition element contacts a distal end of the prosthetic valve and a distal end of the transition element contacts a proximal end of the nosecone.

10. A method, comprising:
advancing a delivery apparatus of a transcatheter delivery system to a target implantation site in a patient, the delivery apparatus including an outer shaft with a distal end portion forming a sheath enclosing a radially compressed prosthetic valve therein, proximate to a proximal end of a nosecone of the delivery apparatus;
after reaching the target implantation site, moving the distal end portion of the outer shaft away from the nosecone, in an axial direction, to uncover the prosthetic valve; and
expanding a transition element of the delivery apparatus in a space formed between the proximal end of the nosecone and a distal end of the prosthetic valve.

11. The method of claim 10, wherein the prosthetic valve expands to a partially expanded state upon moving the distal end portion of the outer shaft away from the nosecone.

12. The method of claim 10, further comprising, after expanding the transition element, repositioning the prosthetic valve at the target implantation site, and further comprising, after repositioning the prosthetic valve, actively expanding, in a radial direction, the prosthetic valve to a radially expanded state, wherein actively expanding the prosthetic valve includes actively expanding the prosthetic valve via one or more actuator assemblies of the delivery apparatus, the one or more actuator assembly extending from an interior of the outer shaft and coupled to the prosthetic valve.

13. The method of claim 10, wherein the transition element is an inflatable balloon, and wherein expanding the transition element includes inflating the inflatable balloon from a deflated state to an inflated state.

14. The method of claim 10, wherein the transition element is a pre-inflated balloon and wherein expanding the transition element includes passively expanding the pre-inflated balloon from a radially compressed state to a radially expanded state, wherein the pre-inflated balloon assumes its pre-inflated size when in the radially expanded state.

15. The method of claim 10, wherein the transition element is a compressible element including one of a compressible foam and a sponge material, and wherein expanding the transition element includes passively expanding the compressible element from a compressed state to an expanded, non-compressed state, wherein the compressible element is in its resting state when in the expanded state.

16. The method of claim 10, wherein the transition element is a mechanical element comprising an expandable frame having a distal end coupled to the nosecone, and wherein expanding the transition element includes expanding a proximal end of the expandable frame from a compressed state to an expanded state.

17. An assembly, comprising
a prosthetic valve; and
a delivery apparatus, comprising:
an outer shaft with a distal end portion forming a sheath adapted to enclose the prosthetic valve therein in a radially compressed configuration;
an inner shaft arranged within the outer shaft and including a nosecone arranged at a distal end of the inner shaft, the nosecone arranged outside of the outer shaft, wherein the outer shaft and the inner shaft are configured to move axially relative to one another to move the nosecone away from the distal end portion of the outer shaft and uncover the prosthetic valve; and
an expandable transition element disposed between the prosthetic valve and the nosecone, the expandable transition element adapted to expand from a non-expanded state within the outer shaft to an expanded state outside the outer shaft, wherein the transition element is in the non-expanded state when the sheath covers the prosthetic valve and the transition element and is in the expanded state when the sheath is moved away from the nosecone to uncover the prosthetic valve and wherein, in the expanded state, the transition element forms a continuous transition from the nosecone to the prosthetic valve.

18. The assembly of claim 17, wherein a distal end of the transition element is attached to a proximal end of the nosecone, wherein the delivery apparatus further comprises at least one actuator assembly arranged within the outer shaft and releasably coupled to the prosthetic valve, and wherein the at least one actuator assembly is configured to radially expand the prosthetic valve.

19. The assembly of claim 17, wherein the transition element is a balloon.

20. The assembly of claim 17, wherein the transition element is a compressible element including one or more of a compressible foam and a sponge, and wherein a proximal end of the compressible element is tapered inward toward a central longitudinal axis of the assembly.
